(19) United States
(12) Patent Application Publication
(10) Pub. No.: US 2010/0218275 A1
(43) Pub. Date: Aug. 26, 2010

(54) METHOD FOR INCREASING SALT TOLERANCE OF PLANT BY OVEREXPRESSING SYFBP/SBPASE GENE ISOLATED FROM SYNECHOCYSTIS AND PLANT PRODUCED BY THE SAME

(75) Inventors: Jang Ryol Liu, Daejeon (KR); Sung Ran Min, Daejeon (KR); Won Joong Jeong, Daejeon (KR); Hwa Jee Chung, Daejeon (KR); Hyun Tae Kim, Daegu (KR); Ju Young Park, Daejeon (KR); Jong Hyun Kim, Daejeon (KR)

Correspondence Address: OSHA LIANG L.L.P., TWO HOUSTON CENTER, 909 FANNIN, SUITE 3500, HOUSTON, TX, 77010, US

(73) Assignee: KOREA RESEARCH INSTITUTE OF BIOSCIENCE AND BIOTECHNOLOGY, YUSEONG-GU, DAEJEON (KR)

(21) Appl. No.: 12/739,316

(22) PCT Filed: Oct. 21, 2008
(86) PCT No.: PCT/KR08/06215

§ 371 Date: Apr. 30, 2010

(30) Foreign Application Priority Data
Oct. 24, 2007 (KR) 10-2007-0107271

(51) Int. Cl. A01H 1/00; A01H 5/00; C07H 21/04; C12N 15/82

(52) U.S. Cl. 800/278; 800/298; 536/23.2; 435/320.1

(57) ABSTRACT

The present invention relates to a method for increasing salt tolerance of a plant by overexpressing SyFBP/SBPase gene isolated from Synechocystis, a plant and seed having increased salt tolerance ability that is produced by the same method, a composition for increasing salt tolerance of a plant in which gene encoding SyFBP/SBPase is comprised, and a recombinant vector for the transformation of chloroplasts in which gene encoding SyFBP/SBPase is comprised.

### TECHNICAL FIELD

The present invention relates to a method for increasing salt tolerance of plant by overexpressing SyFBP/SBPase gene isolated from Synechocystis and a plant and seeds having increased salt tolerance ability that is produced by the same method.

2. Background Art

Synechocystis is the first photoautotroph which appeared at the early stage of formation of earth and is an important bioorganism responsible for the conversion of the practically oxygen-free ancient atmospheric environment into the present-day oxygen-rich atmospheric environment. Synechocystis is also considered as the origin of chloroplasts included in higher plants. Through a photosynthetic reaction, it can biologically synthesize organic substances from water, carbon dioxide and a small amount of inorganic salts by using sunlight as an energy source. As a result, Synechocystis can amplify in a great amount in an autotrophic manner. Further, many species belonging to Synechocystis have an ability to fix nitrogen so that they occupy a key position in the ecological system as helping a nitrogen assimilation of other bioorganisms.

There have been continuous efforts to increase biomass and to improve a crop productivity based on a conventional and a molecular breeding methods. Meanwhile, chloroplasts are known to provide nutrients that are required for survival of a plant, by self-producing a gene for the protein responsible for photosynthesis and a protein which constitutes the chloroplasts (Sinclair, T. R. et al., 2004, Trends Plant Sci. 9, 70-75; Sharma-Natu P. and Ghildiyal, M. C. 2005, Current Science 88, 1918-1928). Photosynthetic reaction in algae and plants that are present on earth occurs in chloroplasts. By consuming ATP and NADPH that are produced during a light reaction process, CO₂ contained in the atmosphere is fixed via Calvin cycle into a form of an initial carbohydrate that is used by a plant. For such Calvin cycle, lots of enzymes are summoned and used. Some of the main enzymes include phosphoribulokinase (PRK), ribulose-1,5-bisphosphate carboxylase/oxygenase (Rubisco), glyceraldehydes-3-phosphate dehydrogenase (GAPDH), chloroplastic fructose-1,6-bisphosphatase (FBPase), sedoheptulose 1,7-bisphosphatase (SBPase) and so on. These enzymes have been extensively studied and identified by using transformed plants (Raines, C. A. 2003, Photosynth. Res. 75, 1-10).

FBPase and SBPase, which are the enzymes involved in Calvin cycle, are mainly responsible for re-synthesis of ribulose 1,5-bisphosphate (RuBP) and production of starch. There has been a report that, when the activity of FBPase is reduced, efficiency of photosynthesis is lowered, plant growth is delayed, potato tuber production and nitrogen metabolism and sucrose production in Arabidopsis are lowered, and also production of tomato is inhibited (Koβmann, J. et al., 1994, Plant J. 6, 637-650; Obiadalla-Ali, H. et al., 2004, Planta 219, 533-540; Sahrawy, M. et al., 2004, J. Exp. Bot. 55, 2495-2503).

Similarly, it is also known that when the activity of SBPase is reduced, a change in carbon assimilation in plant leaves is occurred, resulting in inhibited plant growth (Harrison, E. P. et al., 1998, Planta 204, 27-36; Olcer, H. et al., 2001, Plant Physiol. 125, 982-989; Lawson, T. et al., 2006, Plant Cell Environ. 29, 48-58). In the case of transformed tobacco plant in which SBPase gene from Arabidopsis or Chlamydomonas is overexpressed, efficiency of photosynthetic reaction is increased and also the amount of biologically synthesized sucrose or starch is increased, resulting in overall increase of the plant biomass (Lefebvre, S. et al., 2005, Plant Physiol. 138, 451-460; Tamoi, M. et al., 2006, Plant Cell Physiol. 47, 380-390). Furthermore, transgenic tobacco plants expressing a cyanobacterial (Synechococcus PCC7942) fructose-1,6-/sedoheptulose-1,7-bisphosphatase (FBP/SBPase) and FBPase-II targeted to chloroplasts show enhanced photosynthetic efficiency and growth characteristics under normal atmospheric condition (Miyagawa, Y. et al., 2001, Nat. Biotechnol. 19, 965-969; Tamoi, M. et al., 2006, Plant Cell Physiol. 47, 380-390).

FBPase and SBPase are now known to be a key enzyme for the regulation of Calvin cycle and fragmentation and carbon-carbon bonding. As such, these two enzymes can serve as a very good genetic source for the preparation of a genetically modified plant which is used for increasing biomass of a plant and improving crop productivity.

Meanwhile, when irrigation is performed for the cultivation of crops, concentration of water-soluble salts such as sodium, potassium, calcium, magnesium, sulfate, and chlorine in crop field becomes higher. Once such salts are available in soil more than a certain level, water-absorbing property of a plant via roots is damaged and normal metabolic reaction of the plant cells becomes impossible. Higher the concentration of the salts, less amount of water can be absorbed into the plant. As a result, not only the crop productivity is lowered but also there can be a situation in which the plant itself does not survive at all.

The above-described damage caused by salt is one of the main factors which significantly limit the productivity of crops, and it corresponds to one of the hardest problems to be solved in an agricultural field. According to U.S. Dept. of Agriculture, it is reported that almost 10,000,000 ha of an agricultural field is lost every year over the world due to salt damage caused by irrigation. In order to solve the problems caused by saltification, many researchers have studied to develop salt-tolerant crops by plant breeding such as cross breeding and the like. However, no remarkable results are obtained until now.

Under the circumstances, a new and innovative technique which can be used for inducing salt tolerance in major crops and/or plants is now required. Many researchers are carrying out studies to increase salt tolerance by transforming crops and/or plants with foreign genes. However, it has not been reported until now that salt tolerance of a plant can be improved by overexpression of SyFBP/SBPase gene, which is isolated from Synechocystis, in a plant.

### DETAILED DESCRIPTION OF THE INVENTION

### Technical Problem

According to the present invention, it is determined that salt tolerance of plants can be improved by expressing SyFBP/SBPase gene isolated from Synechocystis in plants.

### Technical Solution

In order to solve the above-described problems, the present invention provides a method for increasing salt tolerance of a plant by overexpressing in the plant SyFBP/SBPase gene isolated from Synechocystis.

Furthermore, the present invention provides a plant and seed having increased salt tolerance that are produced by the above-described method.

Furthermore, the present invention provides a composition for increasing salt tolerance of a plant in which gene encoding SyFBP/SBPase is comprised.

Still furthermore, the present invention provides a recombinant vector for the transformation of chloroplasts in which gene encoding SyFBP/SBPase is comprised.

### ADVANTAGEOUS EFFECTS

According to the present invention, by overexpressing SyFBP/SBPase gene in a plant, salt tolerance of the plant can be improved.

### BRIEF DESCRIPTION OF THE DRAWINGS

FIG. 1 is a schematic drawing of a vector for transforming chloroplast according to the present invention.

FIG. 2 shows the salt tolerance of tobacco plants that are transformed with SyFBP/SBPase according to the present invention. CtVG corresponds to a control vector group. CpFBP-5 and CpFBP-7 correspond to a FBP transformant. The solid bar at the right side represents a relative amount of chloroplasts before the salt treatment. The gray bar represents a relative amount of chloroplasts after the salt treatment.

FIG. 3 shows germination rate of the tobacco plants according to the present invention, that are transformed with SyFBP/SBPase, under salt condition. Panel A shows the results for the case wherein seeds are germinated in MS agar plate which comprises 0-300 mM NaCl. For Panel B, only the plants that had been germinated from the seeds were counted wherein the seeds were maintained for 21 days in MS agar plate comprising NaCl. Data are based on a percentage value of 150 geminated seeds for each line and represent an average of the results obtained from three individual experiments.

FIG. 4 shows root growth of the tobacco plants according to the present invention, that are transformed with SyFBP/SBPase, on a saline containing plate. Each of A, B, C and D represents NaCl concentration of 0, 100, 200, or 300 mM. For 21 days under this NaCl condition, the root length was measured.

FIG. 5 shows the whole plant response by the tobacco plants of the present invention, that are transformed with SyFBP/SBPase, after the NaCl treatment. Panel A corresponds to a phenotype of the plant 14 days after the salt treatment. Panel B shows the recovery of the plant after supply of water. Panel C and D show a result of the analysis of chlorophyll fluorescence.

### BEST MODE FOR CARRYING OUT THE INVENTION

In order to achieve the purpose of the present invention as described in the above, the present invention provides a method for increasing salt tolerance of a plant by overexpressing in the plant SyFBP/SBPase gene isolated from Synechocystis. Said Synechocystis is preferably Synechocystis PCC 6803, and said FBP protein (SyFBP/SBPase) may preferably have an amino acid sequence of SEQ ID NO: 2. More specifically, the present invention provides a method for increasing salt tolerance of a plant comprising a step of overexpressing SyFBP/SBPase gene by transforming the plant cells with a recombinant plant expression vector which comprises a gene encoding FBP protein (SyFBP/SBPase) isolated from Synechocystis PCC 6803.

According to the present invention, in order to obtain a plant which expresses a high amount of the enzymes FBPase and SBPase, transformation by which the gene en coding said proteins is directly expressed in chloroplasts of the plant is carried out. Chloroplast transformation has higher expression efficiency compared to a nuclear transformation method. Further, it is advantageous in that many genes can be expressed together. For achieving such effect, inventors of the present invention isolated a gene encoding FBP protein (SyFBP/SBPase) from Synechocystis PCC 6803. Further, based on the isolated gene, a recombinant plant expression vector was constructed. The protein encoded by the isolated gene has an amino acid sequence of SEQ ID NO: 2. The above-described isolated gene may preferably have a nucleotide sequence of SEQ ID NO: 1. The SyFBP/SBPase gene, that is introduced to the recombinant plant expression vector of the present invention, may further comprise a nucleotide sequence encoding the protein with SyFBP/SBPase activity and having at least 70%, at least 80%, at least 90%, at least 95% homology, or at least 99% homology with the nucleotide sequence of SEQ ID NO: 1, in addition to the nucleotide sequence of SEQ ID NO: 1.

According to a method of one embodiment of the present invention, any kind of plant expression vector that is known in the pertinent art can be used as a recombinant plant expression vector. However, a vector for chloroplast transformation is preferred.

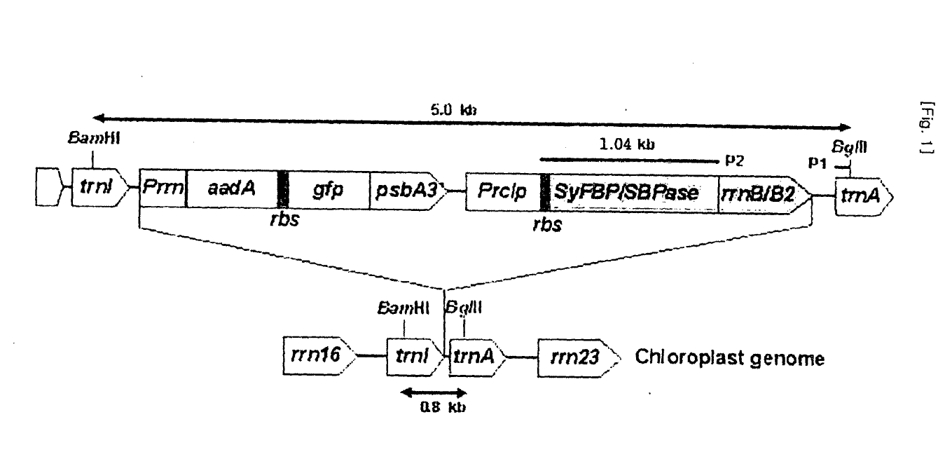

More preferably, it can be CpFBP vector for chloroplast transformation having a cleavage map shown in FIG. 1. Said vector comprises Clp promoter originating from rice and rrnB1/B2 terminator originating from Escherichia coli, and it is inserted into a corresponding region in a genome of a plant chloroplast.

The term “recombinant” indicates a cell which replicates a heterogeneous nucleotide or expresses said nucleotide, a peptide, a heterogeneous peptide, or a protein encoded by a heterogeneous nucleotide. Recombinant cell can express a gene or a gene fragment, that are not found in natural state of cell, in a form of a sense or antisense.

In addition, a recombinant cell can express a gene that is found in natural state, provided that said gene is modified and re-introduced into the cell by an artificial means.

The term “vector” is used herein to refer DNA fragment (s) and nucleotide molecules that are delivered to a cell. Vector can be used for the replication of DNA and be independently reproduced in a host cell. The terms “delivery system” and “vector” are often interchangeably used. The term “expression vector” means a recombinant DNA molecule comprising a desired coding sequence and other appropriate nucleotide sequences that are essential for the expression of the operatively-linked coding sequence in a specific host organism. Promoter, enhancer, termination signal and terminator that can be used for an eukaryotic cell are all publicly well known.

Expression vector preferably comprises at least one selective marker. Said selective marker is a nucleotide sequence having a property that it can be selected by a common chemical method. Every gene which can be used for the differentiation of transformed cells from non-transformed cell can be a selective marker. Example includes, a gene resistant to herbicides such as glyphosate and phosphintricin, and a gene resistant to antibiotics such as kanamycin, G418, bleomycin, hygromycin, and chloramphenicol, but not limited thereto.

For the plant expression vector according to one embodiment of the present invention, a promoter can be any of CaMV 35S, actin, ubiquitin, pEMU, MAS or histone promoter, or Clp promoter originating from rice. Clp promoter originating from rice that can be used for chloroplast transformation is preferred. The term “promoter” means a DNA molecule to which RNA polymerase binds in order to initiate its transcription, and it corresponds to a DNA region upstream of a structural gene. The term “plant promoter” indicates a promoter which can initiate transcription in a plant cell. The term “constitutive promoter” indicates a promoter which is active in most of environmental conditions and development states or cell differentiation states.

For the above-described terminator, any conventional terminator can be used for the present invention. Example includes, nopaline synthase (NOS), rice α-amylase R Amy1 A terminator, phaseoline terminator, and a terminator for octopine gene of Agrobacterium tumefaciens, rrnB1/B2 terminator from Escherichia coli and the like. Preferably, it is rrnB1/B2 terminator from Escherichia coli.

Plant transformation means any method by which DNA is delivered to a plant. Such transformation method does not necessarily have a period for regeneration and/or tissue culture. Transformation of plant species is now quite general not only for dicot plants but also for monocot plants. In principle, any transformation method can be used for introducing a hybrid DNA of the present invention to an appropriate progenitor cells. It can be appropriately selected from a calcium/polyethylene glycol method for protoplasts (Krens, F. A. et al., 1982, Nature 296, 72-74; Negrutiu I. et al., June 1987, Plant Mol. Biol. 8, 363-373), an electroporation method for protoplasts (Shillito R. D. et al., 1985 Bio/Technol. 3, 1099-1102), a microscopic injection method for plant components (Crossway A. et al., 1986, Mol. Gen. Genet. 202, 179-185), a particle bombardment method for various plant components (DNA or RNA-coated) (Klein T. M. et al., 1987, Nature 3 27, 70), or a (non-complete) viral infection method in Agrobacterium tumefaciens mediated gene transfer by plant invasion or transformation of fully ripened pollen or microspore (EP 0 301 316), etc.

The method of the present invention comprises a step of transforming a plant cell with the recombinant vector according to the present invention. The transformation can be performed by a particle bombardment after gold particles are coated with the recombinant vector of the present invention. In addition, the method of the present invention may also comprise a step of re-differentiating a transformed plant from the above-mentioned transformed plant cells. The method of re-differentiating a transformed plant from the transformed plant cells can be carried out by using any method that is publicly known in the pertinent art.

According to the method of one embodiment of the present invention, the plant includes monocot and dicot plants. Examples of monocot plant include rice, wheat, barley, bamboo shoot, corn, taro, asparagus, onion, garlic, scallion, leek, wild rocambole, hemp, and ginger, but not limited thereto. Examples of dicot plant include, tobacco, Arabidopsis, eggplant, pepper, tomato, potato, burdock, crown daisy, lettuce, Chinese bellflower, spinach, chard, sweet potato, celery, carrot, coriander, parsley, Chinese cabbage, cabbage, leaf mustard, radish, watermelon, melon, cucumber, zucchini, gourd, strawberry, soy bean, mung bean, kidney bean, sweet pea and the like, but not limited thereto. Tobacco is preferred.

In order to achieve another purpose of the invention, the present invention provides a plant having increased salt tolerance that is produced by the method of the present invention. More specifically, the plant having salt tolerance according to the present invention can be produced by transforming a plant with a recombinant vector comprising SyFBP/SBPase gene, followed by induction of shoots, root growth and soil acclimatization according to a conventional method. That is, a plant fragment that has been transformed with the recombinant vector comprising SyFBP/SBPase gene is placed on an appropriate medium known in the pertinent art, followed by cultivating it under proper condition to induce shoots. Once the shoots are formed, the plant is transferred to a hormone-free medium and cultivated again. After approximately 2 weeks, thus-obtained shoots are transferred to a medium for inducing root growth so that the roots can be formed. Once the roots are induced, the plant is transplanted in soils and acclimatized to obtain a plant having salt tolerance. Preferably, said plant is tobacco.

Further, the present invention provides the seeds of a plant having increased salt tolerance.

Further, the present invention provides a composition for increasing salt tolerance of a plant in which a gene encoding FPB protein (SyFBP/SBPase) that is isolated from Synechocystis PCC 6803 is comprised. Said gene may preferably have a nucleotide sequence of SEQ ID NO: 1. By expressing said gene in a corresponding plant with transformation, salt tolerance of the plant can be improved.

Still further, the present invention provides a recombinant vector for the transformation of chloroplasts in which a gene encoding FPB protein (SyFBP/SBPase) that isolated from Synechocystis PCC 6803 is comprised. Said gene may preferably have a nucleotide sequence of SEQ ID NO: 1. Preferably, said vector can be CpFBP vector which has a cleavage map shown in FIG. 1. However, it is not limited thereto.

The present invention will now be described in greater detail with reference to the following examples. However, it is only to specifically exemplify the present invention and in no case the scope of the present invention is limited by these examples.

### EXAMPLES

### Experimental Method

1. Construction of a Vector for the Transformation of Chloroplasts

FBP/SBPase gene was obtained from genomic DNA of Synechocystis PCC6803 by PCR amplification Method Using the Primer 5′-GAG CTC AGG AGG TAT ACA GTG GAC AGC ACC CTC GGT-3′ (SEQ ID NO: 3) (SacI site is underlined) and the primer 5′-CTG CAG TTA ATG CAG TTG GAT TAC TTT GGG G-3′ (SEQ ID NO: 4) (PstI site is underlined). The gene obtained from said amplification was cloned in pGEM-T Easy (Promega, Madison, Wis.) and its nucleotide sequence was identified. The FBP/SBPase with identified nucleotide sequence was digested with restriction enzymes of SacI/PstI and then sub-cloned in RcIpADGHt. The resultant was named as Rclp-SyFBP/SBP. The subcloned Rclp-SyFBP/SBP was digested with the restriction enzymes of XhoI/EcoRI, and ligated as a blunt to PstI site of CtVG, which is a vector for the transformation of chloroplasts. As a result, CpFBP, which is a vector for the transformation of chloroplasts, was obtained.

2. Conditions for the Transformation and Cultivation of Plants

The method of transforming chloroplasts of a tobacco plant (Nicotiana tabacum L. cv. Samsun) is the same as the one described in the Korean Patent Registration No. 468624. Specifically, seeds of the wild type tobacco plant (Nicotiana tabacum, cv. Samsun) were germinated in an incubator for 8 weeks. Then, from the young plants, leave were harvested and placed on MS medium to which 1 mg/L BAP and 0.1 mg/L NAA have been added for the plastid transformation. The vector for the plastid transformation, which has been prepared in the above, was coated on gold particles having diameter of 0.6 μm by using CaCl₂ and spermidine, followed by plastid transformation using the PDS-1000/He gene delivery system manufactured by Bio-Rad (Hercules, Calif.) under the condition including acceleration power of 1,100 psi, target distance of 9 cm and a pressure of 28 in/Hg (i.e., under vacuum).

All the analysis was carried out using a transformed T₁ plant. The control plant and the transformant were placed in a basic MS medium comprising 2% sucrose. They were then germinated with lighting cycle of 16-hour light/8-hour dark. After 5 weeks, they were planted in soils and cultivated in greenhouse during summer days (800-1600 μmol·m⁻² sec⁻¹, 25-35° C.).

3. Southern analysis and Northern analysis Whole genomic DNA was separated from the tobacco leaves by using DNeasy Plant Mini Kit (Qiagen, Hilden, Germany). About 4 μg of the genomic DNA was digested with BamHI and BglII, subjected to electrophoresis on 1 agarose gel, and then transferred to a Zeta-Probe GT Blotting Membrane (Bio-Rad, Hercules, Calif.). BamHI-BglII DNA fragment (0.6 kb, P1 probe), which comprises trnl contained the genome of the plastid, was labeled with the radioactive isotope [³²P] dCTP to confirm that aadA and gfp have been successfully inserted therein. The pre-hybridization and hybridization processes were carried out in a 0.25 M sodium phosphate buffer (pH 7.2) comprising 7% (w/v) SDS for 16 hours at 65° C. After washing twice with 0.2 M sodium phosphate buffer (pH 7.2) comprising 5% (w/v) SDS for 30 minutes at 65° C., it was subjected to a reaction on X-ray film for 3 hours for the confirmation.

By using Trizol reagent (Invitrogen, Carlsbad, Calif.), whole RNAs were extracted from the tobacco leaves. Thus-obtained whole RNAs (2 μg) were electrophoresed using 1.2% agarose gel comprising 5.1% (v/v) formaldehyde. Then, the RNAs were transferred to Zeta-Probe GT Blotting Membrane (Bio-Rad, Hercules, Calif.), and then the hybridization was carried out by labeling the FBP/SBP gene fragment (i.e., P2 probe) with [³²P] dCTP.

4. Measurement of the Amount of Chloroplasts

Leaf pieces (1.13 cm²) were ground in liquid nitrogen by using a mortar. The a mount of chlorophyll a and chlorophyll b were measured according to the method suggested by Jeong et al (Jeong, S. W. et al., 2002, Mol. Cells, 13, 419-428).

The experimental procedures that are not specifically described herein can be carried out according to a general molecular biological method that is well known in the pertinent art.

### Example 1

### Selection of a Vector for Chloroplast Transformation and a Transformed Tobacco Plant

Within the vector for chloroplast transformation, a nucleotide sequence region has been inserted for homologous recombination. For the expression of a selection marker, Prrn promoter and psbA 3′ UTR were utilized. For the expression of FBP/SBPase (slr2094) gene, which has been found from Synechocystis spp. PCC 6803, Clp promoter and rrnB1/B2 terminator, separated from rice and Escherichia coli, respectively, were used (see FIG. 1). Five individual transformed tobacco plants T₀ (CpFBP-1, -2, -5, -7, and -8) were subjected to Southern blot analysis to confirm that the foreign gene has been successfully inserted into the plant. Seeds of the T₀ plant were germinated in a selection medium which comprises spectinomycin as antibiotics for selection. Thus-obtained T1 generation was confirmed again by Southern blot analysis. As a result, a band that is the same as that of T₀ plant was observed, suggesting that the FBP/SBPase gene as a foreign gene has been indeed delivered to the next generation. All of the CpFBP T₁ plants were analyzed with Northern blot analysis to confirm that FBP/SBPase (1.8 kb) gene is expressed together with several thick bands. At the same time, it was also confirmed that from CtVG, which is the control plant, no such expression was observed. It is considered that the thick bands having different size are presumably the results of the expression of rrn16 present inside the genome of the chloroplast or Prrn promoter of the vector.

### Example 2

### Determination of Salt Tolerance of the Tobacco Plant which has Been Subjected to Chloroplast Transformation with SyFBP

1. Amount of the Chloroplast

CpFBP-5 and CpFBP-7 seeds of which chloroplasts have been transformed with FBP (SyFBP/SBPase) gene isolated from Synechocystis PCC 6803 and T1 seeds of CtVG control vector group were sterilized and placed in a medium to which MSBM500Sp (MS basal medium+3% sucrose+500 mg/L spectinomycin+0.6% phytoagar) has been added, followed by incubating them for 2 weeks at 25° with lighting condition of cool-white fluorescence of about 40 μmol·m⁻²·sec⁻¹. After transplanted in the MSBM liquid medium comprising 0.250 mM NaCl, the seeds were incubated for five days and the amount of the chloroplast was measured.

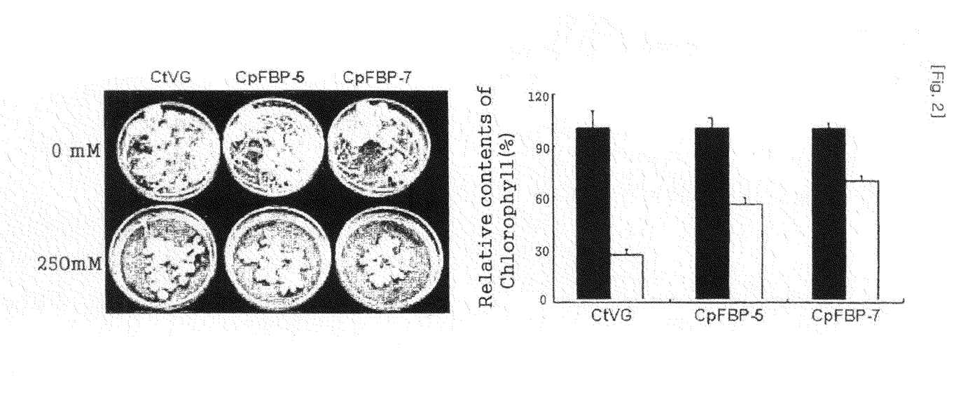

When the amount of the chloroplast was measured after the salt treatment, it was found that the tobacco plant which has been transformed with SyFBP/SBPase has higher tolerance to salt compared to the control group plant comprising CtVG vector (see FIG. 2). In FIG. 2, CtVG corresponds to the control vector group while CpFBP-5 and CpFBP-7 correspond to the FBP transformants. In the graph of FIG. 2, the solid bar at the right side represents a relative amount of chloroplasts before the salt treatment. The gray bar represents a relative amount of chloroplasts after the salt treatment. As it can be seen from the graph, it was confirmed that in the control vector group the amount of chloroplast was reduced significantly after the salt treatment while in the transformants of the present invention the reduced chloroplast amount was relatively small.

Therefore, it is evident that the tobacco plant which has been transformed with SyFBP/SBPase gene of the present invention has improved salt tolerance compared to the control vector group.

2. Germination Rate

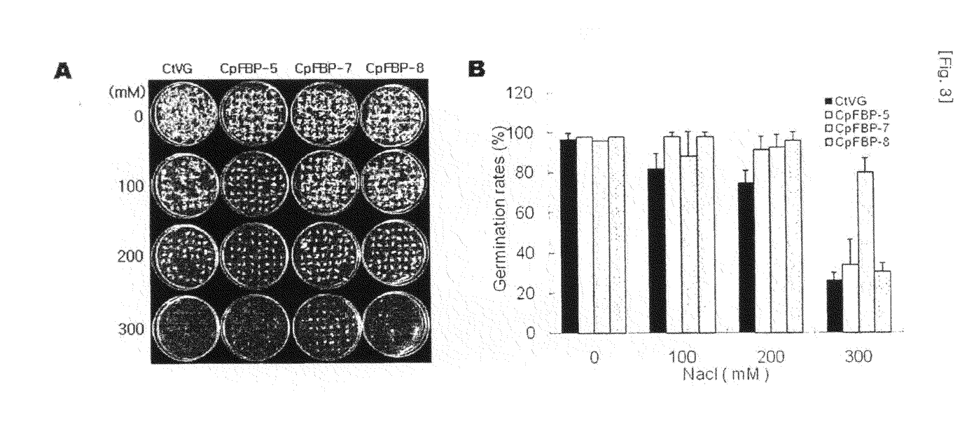

T1 seeds of CpFBP-5, CpFBP-7, and CpFBP-8 of which chloroplasts have been transformed with FBP (SyFBP/SBPase) gene isolated from Synechocystis PCC 6803 and T1 seeds of CtVG control vector group were surface-sterilized by washing them with 70% ethanol for 30 seconds and 0.5% (v/v) sodium hypochlorite solution (NaOCl) for 15 minutes. Then, the seeds were placed in MS basal medium to which 0, 100, 200 or 300 mM NaCl has been added. After incubating them for 21 days at 25° with lighting condition of cool-white fluorescence of about 40 μmol·m⁻²·sec⁻¹, germination rate was determined. The experiments were carried out in triplicate with 150 seeds per each line (see FIG. 3). In FIG. 3, CpFBP-5, CpFBP-7 and CpFBP-8 represent the FBP chloroplast transformant while CtVG represents the control vector group.

As a result of determining germination rate of the tobacco seeds for different concentration of NaCl, it was found that chloroplast transformant of the present invention has higher germination rate at high salt concentration compared to the control vector group. For the basal medium free of any salt or the medium comprising 100 mM NaCl, there is no big difference in germination rate between the chloroplast transformant of the present invention and the control vector group. However, for the group treated with 200 mM NaCl, the chloroplast transformant of the present invention has the germination rate almost 1.3 times higher than that of the control vector group. For the group treated with 300 mM NaCl, the chloroplast transformant of the present invention has the germination rate almost 3 times higher than that of the control vector group. In addition, in terms of plant growth, it was observed that the control vector group which had been germinated at 200 mM salt concentration could not survive after the germination, while the chloroplast transformant of the present invention showed continuous growth.

3. Root Length

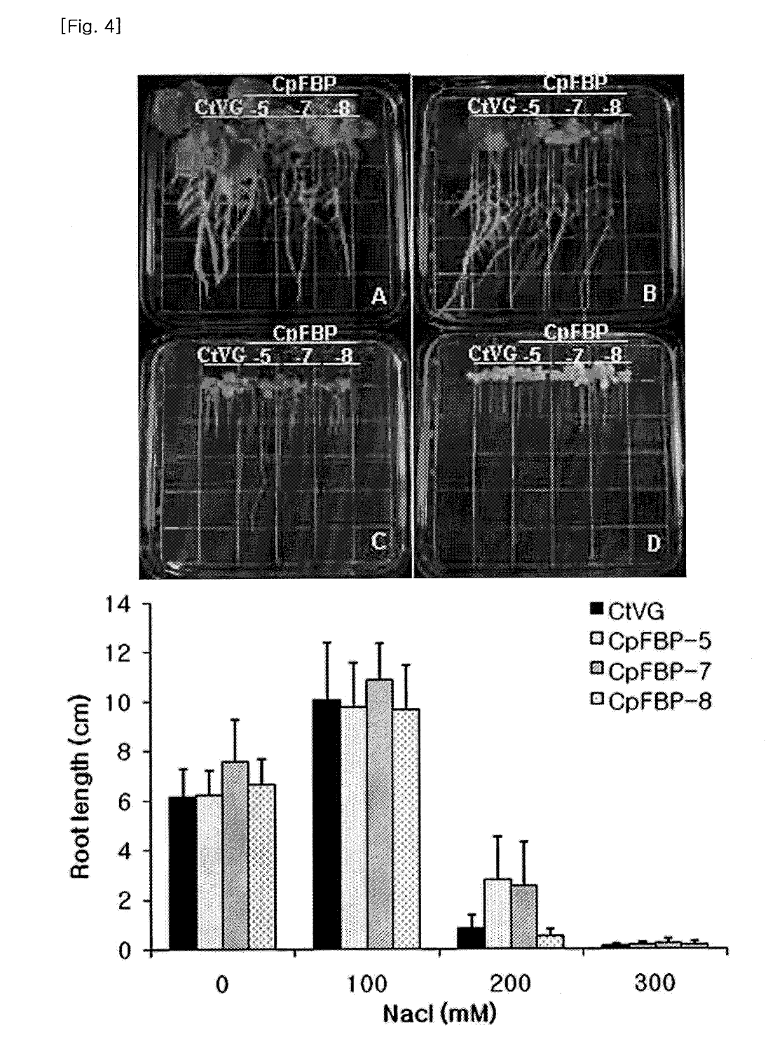

Seeds were sterilized in the same condition as the above-described germination rate test, and then placed in MS basal medium. After 21 days of the incubation, the germinated plants were transferred to MS basal medium (solid) comprising different concentration of salt (i.e., 0, 100, 200 or 300 mM NaCl). After cultivating them for three weeks, root length of the plants was measured (see FIG. 4). In FIG. 4, CtVG represents the control vector group while CpFBP-5, CpFBP-7 and CpFBP-8 represent the FBP transformants.

Specifically, the plants which had been germinated for three weeks in an incubator were transferred to the medium comprising salt, cultivated for three weeks, and then their root length was measured. Although there was no significant difference up to 100 mM salt concentration, it was confirmed that for 200 mM salt treatment group the roots were not successfully formed for the control vector group. Further, it was also observed that the chloroplasts of the control vector group were disrupted due to the presence of salt and the leaves did not grow and turned yellow. On the other hand, the chloroplast transformants of the present invention showed that main roots were successfully developed and even the side roots tend to increase, and the leaves maintained healthy and green color.

4. Whole Plant

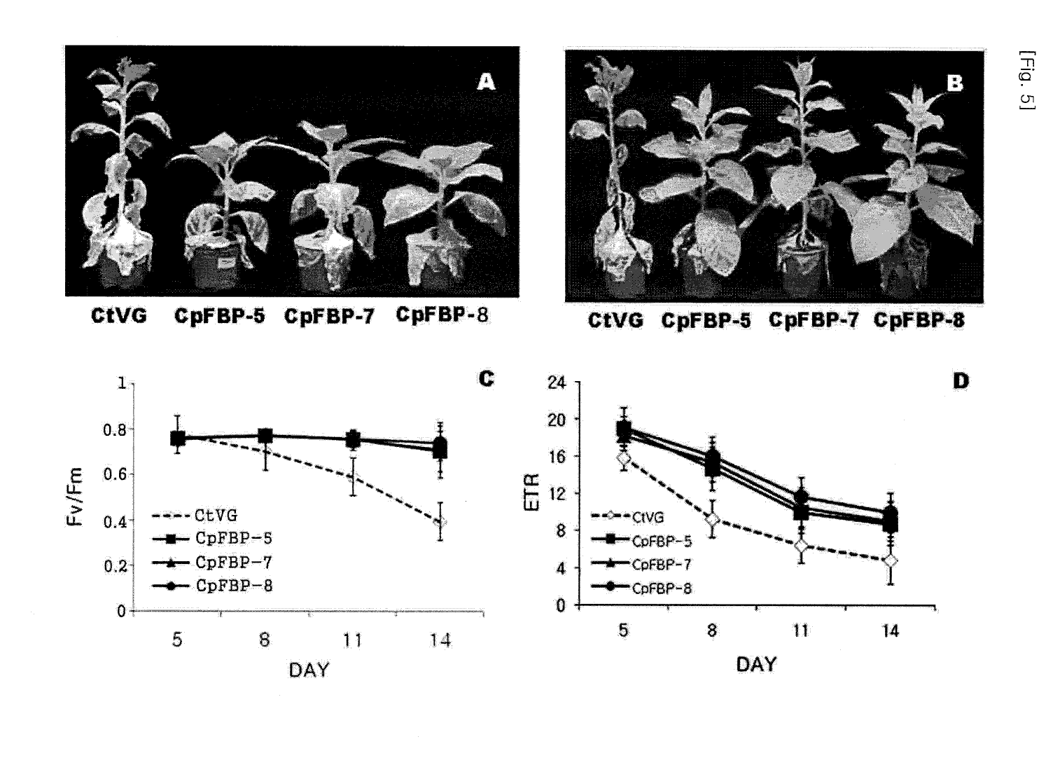

The germinated plants after cultivating them for 21 days in MS basal medium (so lid) were subjected to soil acclimatization and then cultivated again for 8 weeks. Then, the plants were treated with 300 mM NaCl solution for fourteen days, followed by watering the plants on the Day 15 to observe the recovery of the plants. The amount of photosynthesis was measured every three days (see FIG. 5). In FIG. 5, CtVG represents the control vector group while CpFBP-5, CpFBP-7 and CpFBP-8 represent the FBP chloroplast transformants. ETR represents Electron Transport Rate.

The control group showed no significant change until the Day 5. However, starting from the Day 8, the chloroplasts were disrupted and the yellowing phenomenon was observed. Further, Fv/Fm value started to decrease and from the Day 14 withering of the leaves becomes serious. On the other hand, the chloroplast transformants of the present invention showed little change. Specifically, when the plant recovery was observed after water was supplied to the plants from the Day 15, the control vector group continued to wither while the chloroplast transformants of the present invention showed quick recovery and continued to grow.

1. A method for increasing salt tolerance of a plant comprising a step of overexpressing SyFBP/SBPase gene by transforming a plant cell with a recombinant plant expression vector which comprises a gene encoding FBP protein (SyFBP/SBPase) isolated from Synechocystis PCC 6803.

2. The method according to claim 1, which is characterized in that said gene has a nucleotide sequence of SEQ ID NO: 1.

3. The method according to claim 1, which is characterized in that said recombinant plant expression vector is a vector for the transformation of chloroplasts.

4. The method according to claim 1, which is characterized in that said plant is selected from a group consisting of tobacco, Arabidopsis, eggplant, pepper, tomato, potato, burdock, crown daisy, lettuce, Chinese bellflower, spinach, chard, sweet potato, celery, carrot, coriander, parsley, Chinese cabbage, cabbage, leaf mustard, radish, watermelon, melon, cucumber, zucchini, gourd, strawberry, soy bean, mung bean, kidney bean, and sweet pea.

5. A plant having increased salt tolerance that is produced by the method according to claim 1.

6. Seeds of the plant according to claim 5.

7. A composition for increasing salt tolerance of a plant, which comprises a gene encoding FBP protein (SyFBP/SBPase) isolated from Synechocystis PCC 6803.

8. The composition according to claim 7, which is characterized in that said gene has a nucleotide sequence of SEQ ID NO: 1.

9. A recombinant vector for the transformation of chloroplasts, which comprises a gene encoding FBP protein (SyFBP/SBPase) isolated from Synechocystis PCC 6803.

10. The recombinant vector for the transformation of chloroplasts according to claim 9, which is characterized in that said vector is CpFBP vector having a cleavage map described in FIG. 1.

11. A plant having increased salt tolerance that is produced by the method according to claim 2.

12. A plant having increased salt tolerance that is produced by the method according to claim 3.

13. A plant having increased salt tolerance that is produced by the method according to claim 4.

14. Seeds of the plant according to claim 11.

15. Seeds of the plant according to claim 12.

16. Seeds of the plant according to claim 13.
